(19) United States
(12) Patent Application Publication
(10) Pub. No.: US 2024/0350283 A1
(43) Pub. Date: Oct. 24, 2024

(54) JOINT PROSTHESIS

(71) Applicants: FONDAZIONE ISTITUTO ITALIANO DI TECNOLOGIA, Genova (IT); I.N.A.I.L. ISTITUTO NAZIONALE PER L'ASSICURAZIONE CONTRO GLI INFORTUNI SUL LAVORO, Roma (IT)

(72) Inventors: Samuel STEDMAN, Genova (IT); Nicolò BOCCARDO, Genova (IT); Lorenzo LOMBARDI, Genova (IT); Matteo LAFFRANCHI, Genova (IT); Lorenzo DE MICHIELI, Genova (IT); Andrea MARINELLI, Genova (IT); Emanuele GRUPPIONI, Genova (IT)

(21) Appl. No.: 18/682,552

(22) PCT Filed: Jul. 28, 2022
(86) PCT No.: PCT/IB2022/056982

§ 371 Date: Feb. 9, 2024

(30) Foreign Application Priority Data
Aug. 9, 2021 (IT) 102021000021563

(51) Int. Cl. A61F 2/58; A61F 2/50

(52) U.S. Cl. CPC A61F 2/582; A61F 2002/5038; A61F 2002/5073; A61F 2002/5093

(57) ABSTRACT

It is provided a joint prosthesis (1) configured to reciprocally rotate a first prosthesis (1a) and a second prosthesis (1b) and comprising: a first attachment (2) to the first prosthesis (1a); a second attachment (3) to the second prosthesis (1b): a hinge (4) defining an axis of rotation (4a) between the attachments (2, 3); a mover (5) configured to apply to the hinge (4) a driving torque defining a reciprocal rotation between the attachments (2, 3); and a compensator (6) configured to apply to the hinge (4) a variable locking torque depending on the angular opening between the attachments (2, 3) and defining the minimum torque to be applied to the hinge (4) to have said reciprocal rotation.

The present invention relates to a joint prosthesis of the type specified in the preamble to the first claim.

In particular, the invention introduces an active joint prosthesis configured to control a rotation of a robotic limb (in particular a portion of an upper limb such as an arm) relative to a second element such as a torso or a limb (in particular a portion of an upper limb such as a forearm). Preferably the joint prosthesis is a prosthetic elbow. Most joint prostheses (also known as prosthetic joints) and in particular the prosthetic elbows currently available are cosmetic or passive and require the user to reposition unnatural and poorly intuitive operations. For example, their repositioning can be performed manually using the other arm or require the patient to move the shoulder in a coordinated pattern.

There are currently few prosthetic joints and therefore few prosthetic elbows. They involve the use of a motor that uses a special transmission to drive a rotation of the joint prosthesis.

An example of an active joint prosthesis is described in U.S. Pat. No. 6,361,570B1. It presents an endo-skeletal joint prosthesis comprising a first component defining an upper limb; an endless screw attached to said first component; and a second component defining a housing for a sprocket engaged to the endless screw; and a motor which rotates the sprocket on the endless screw to drive a rotation between the first component and the second component.

The known technique described includes some major drawbacks.

In particular, known prostheses do not provide the patient with an experience at least comparable to that of the missing limb as they function in an unnatural and unintuitive way.

Another drawback, particularly with active joint prostheses, is that they have poor reliability, high consumption, heavy weight and high transmission noise.

Other drawbacks of these prostheses are the difficulty of control, which, together with the imperfect transmission, lead to delays in moving the elbow with respect to the user's intention.

A not insignificant drawback is the high cost of known active joint prostheses.

In this situation, the technical task at the basis of the present invention is to devise a joint prosthesis capable of substantially obviating at least some of the aforementioned drawbacks.

Within this technical task, it is an important aim of the invention to obtain a reliable prosthesis capable of: functioning naturally, intuitively recognising the user's intention, and providing the patient with an experience as close as possible to the missing limb.

One aim is to achieve a joint prosthesis with a responsive and intuitive control strategy.

Another important aim of the invention is to make a prosthesis that is affordable and comfortable to wear.

Another important aim of the invention is to make a joint prosthesis that produces little noise and has low power consumption.

The specified technical task and purposes are achieved by a joint prosthesis as claimed in the attached claim 1. Examples of preferred embodiments are described in the dependent claims.

The features and advantages of the invention are clarified below by a detailed description of preferred embodiments of the invention, with reference to the accompanying drawings, in which

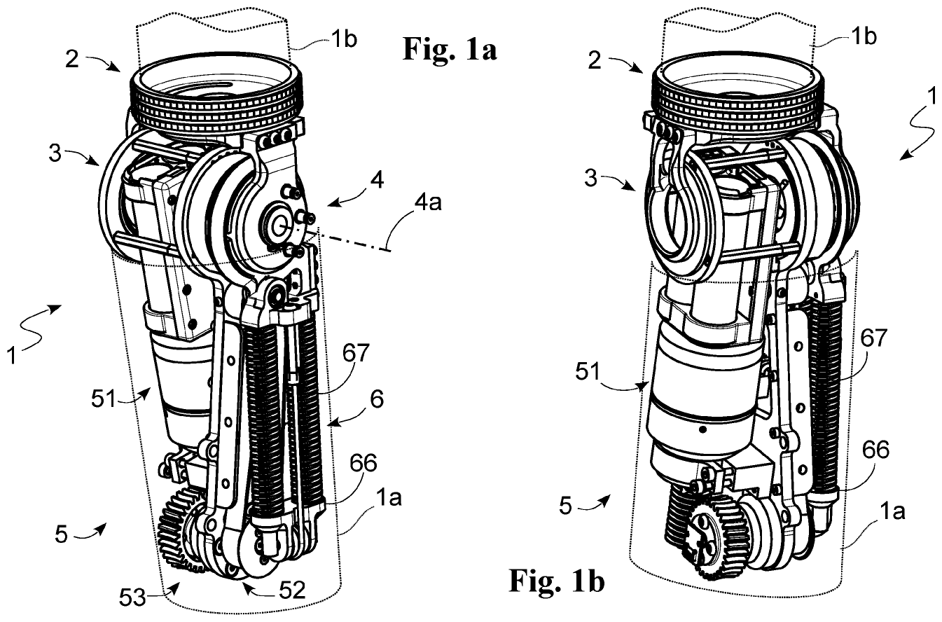

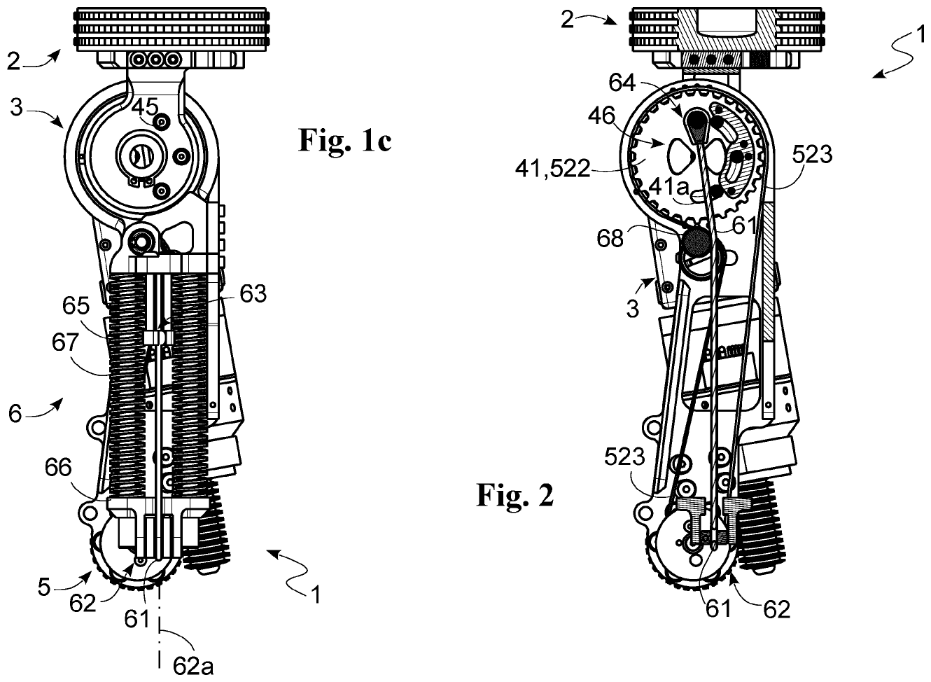

the FIG. 1 shows, in scale, a joint prosthesis according to the invention;

the FIG. 1b illustrates, in scale, a second view of the prosthesis in FIG. 1;

the FIG. 1c shows, in scale, a third view of the prosthesis in FIG. 1;

the FIG. 2 shows a cross-section of the joint prosthesis of FIG. 1c;

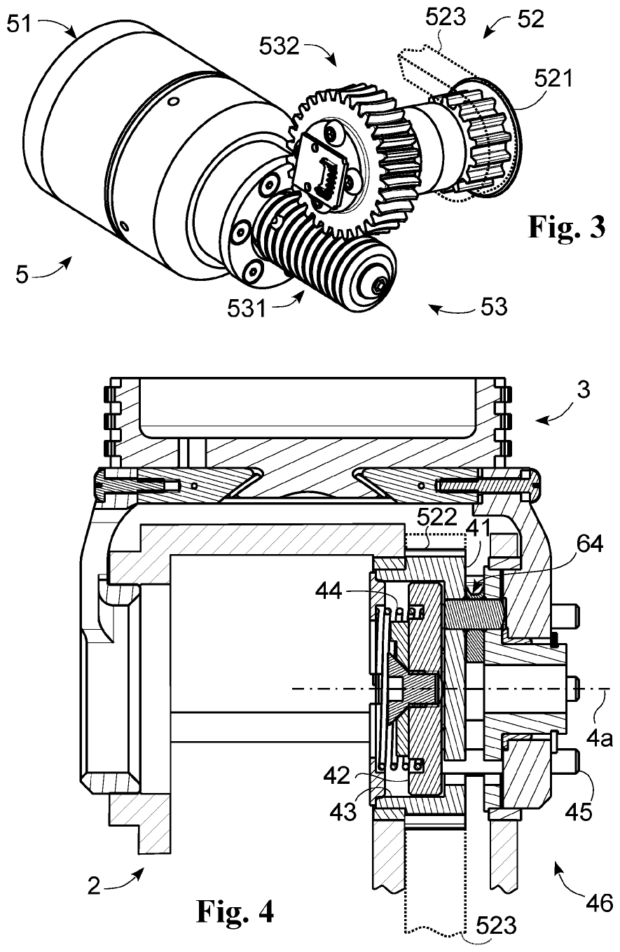

the FIG. 3 shows, in scale, a section of a joint prosthesis assembly according to the invention;

the FIG. 4 shows, in scale, a second joint prosthesis assembly;

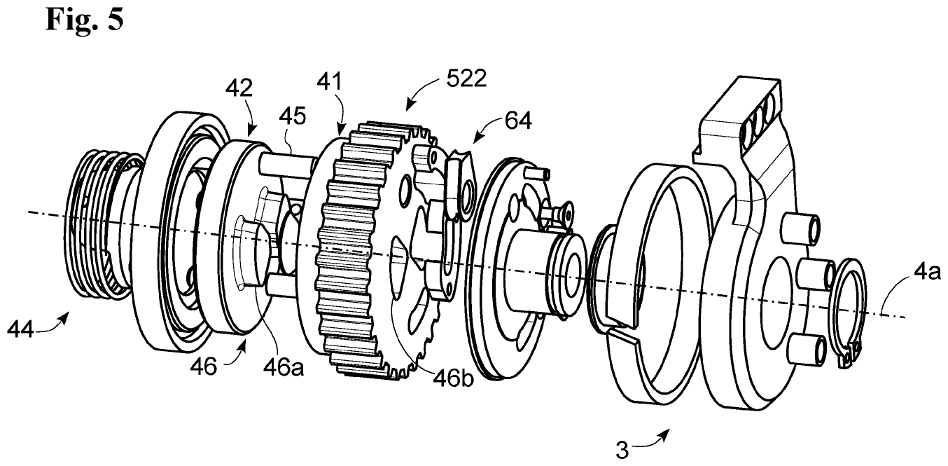

the FIG. 5 reproduces, in scale, an exploded view of the second assembly of FIG. 4;

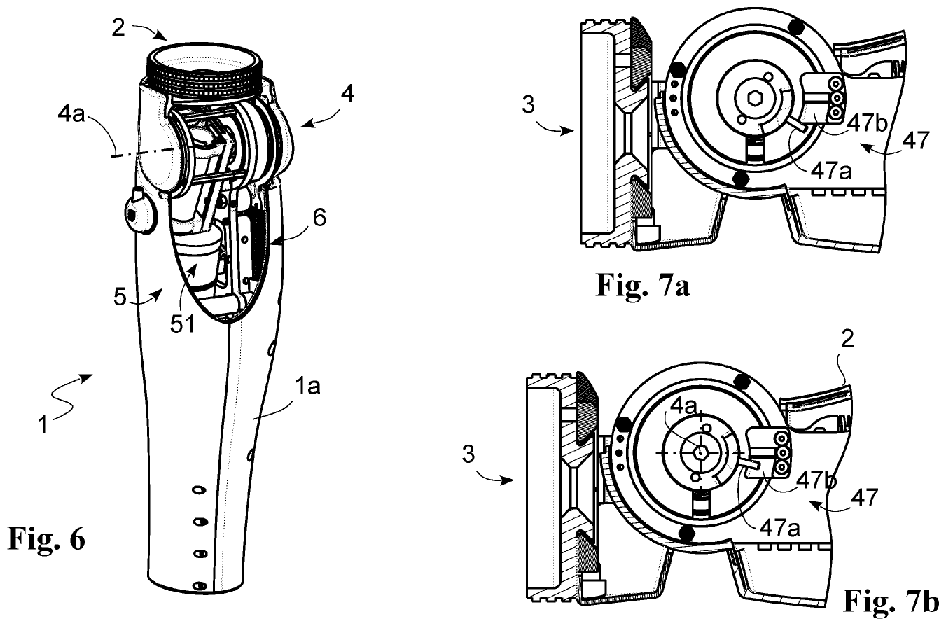

the FIG. 6 shows, in scale, a joint prosthesis according to the invention; and

the FIGS. 7a-7b depict, in scale, a sectional portion of the prosthesis.

In the present document, the measurements, values, shapes and geometric references (such as perpendicularity and parallelism), when associated with words like “about” or other similar terms such as “approximately” or “substantially”, are to be considered as except for measurement errors or inaccuracies due to production and/or manufacturing errors, and, above all, except for a slight divergence from the value, measurements, shape, or geometric reference with which it is associated. For instance, these terms, if associated with a value, preferably indicate a divergence of not more than 10% of the value.

Moreover, when used, terms such as “first”, “second”, “higher”, “lower”, “main” and “secondary” do not necessarily identify an order, a priority of relationship or a relative position, but can simply be used to clearly distinguish between their different components.

The measurements and data reported in this text are to be considered, unless otherwise indicated, as performed in the International Standard Atmosphere ICAO (ISO 2533:1975).

Unless otherwise specified, as results in the following discussions, terms such as “treatment”, “computing”, “determination”, “calculation”, or similar, refer to the action and/or processes of a computer or similar electronic calculation device that manipulates and/or transforms data represented as physical, such as electronic quantities of registers of a computer system and/or memories in, other data similarly represented as physical quantities within computer systems, registers or other storage, transmission or information displaying devices.

With reference to the Figures, the joint prosthesis according to the invention is globally indicated by the number 1.

It is configured to command a reciprocal rotation between a first prosthesis 1a and a second prosthesis 1b. Preferably the joint prosthesis 1 is an elbow allowing rotation with respect to the forearm prosthesis and the arm stump.

The first prosthesis 1a may be a suitably superior robotic limb and in particular a forearm or hand.

The second prosthesis 1b may be identified in a torso or a limb (particularly a portion of an upper limb such as an arm). In some, the second prosthesis 1b may be a prosthetic socket.

The joint prosthesis 1 can define an angle of rotation, i.e. a maximum amplitude of rotation that can be performed by the joint prosthesis 1 itself. This angle can be almost less than 360°, in detail 180°, and appropriately between 60° and 180°.

The joint prosthesis 1 may comprise a first attachment 2 of the joint prosthesis 1 to the first prosthesis 1a; suitably a second attachment 3 of the joint prosthesis 1 to the second prosthesis 1b; a hinge 4 configured to define an axis of rotation 4a between the attachments 2 and 3 and thereby between the prosthesis 1a and 1b; and a mover 5 configured to apply a driving torque defining a reciprocal rotation between the attachments 2 and 3 and thereby between the prostheses 1a and 1b suitably about the axis of rotation 4°.

The hinge 4 can include a clutch defining the maximum torque that can be transmitted from the mover 5 to the second coupling 3, allowing said mutual rotation between couplings 2 and 3.

The clutch (FIG. 3) may comprise a first plate 41; a second plate 42; at least one guide 43 defining a sliding axis between plates 41 and 42 suitably parallel to the axis of rotation 4a, and a spring 44 configured to define, along said guide 43, a reciprocal clamping force between plates 41 and 42 defining said maximum torque. The clutch can define for prosthesis 1 a free configuration in which the second plate 42 is not pressed against the first plate 41 preventing a passage of motion between mover 5 and second attachment 3 and thus attachments 2 and 3 can rotate reciprocally in idle mode; and an operating configuration in which the second plate 42 is pressed against the first plate 41 allowing a passage of motion between mover 5 and second coupling 3 and thus a maximum torque.

The first plate 41 can be integral with the mover 5 so as to be pulled by it in rotation about the axis 4a.

The spring 44 can be configured to push the second plate 42 against the first plate 41.

It can be pre-loaded.

The second plate 42 can be tied to the second coupling 3 so that it can be dragged in rotation around the axis 4a and translate along said guide 43 with respect to it.

The first plate 41 may have a tumbler shape defining a housing for the second plate 42. The side walls of said tumbler may identify the guide 43.

The first plate 41 may come between the second plate 42 and the second connection 3. Consequently, the clutch may include one or more connectors 45 (preferably three connectors 45) from the second plate 42 to the second connection 3 conveniently via the first plate 41

The connector 45 can be a defining pin called a guide 44 for the translation of the second plate 42 along the axis of rotation 42a.

The clutch can define an idle rotation between plates 41 and 42 not exceeding an idle angle.

This idle angle thus identifies the limits of movement of joint prosthesis 1 during the free swing mode defined by said idle rotation. It may be less than 45° and in detail substantially between 45° and 0° and in detail between 30° and 0°.

For this purpose, the first plate 41 may comprise, for each connector 45, a slot 41a having an angular width substantially equal to said idle angle and in which said pin slides during a reciprocal rotation between the plates 41 and 42.

Said idle rotation between the plates 41 and 42 preferably only occurs in free configuration. Accordingly, the clutch may comprise a rotational lock 46 configured to prevent relative rotation between the plates 41 and 42 only in the operating configuration.

The rotational lock 46 only prevents said relative rotation. Therefore, it does not prevent a reciprocal translation of the plates along rotation axis 4a.

The rotational block 46 may comprise a tooth 46a integral with the second plate 42 and a seat 46b for said tooth obtained in the first plate 41.

The seat 46b is counter-shaped to tooth 46a so that when tooth 46a is in seat 46b, rotational lock 46 prevents relative rotation between plates 41 and 42.

The tooth 46a protrudes from the second plate 42 along the axis of rotation 4a towards the first plate 41 appropriately by an extension substantially less than the distance between the plates in free configuration. Consequently, in free configuration the tooth 46a is totally external to the seat 46b allowing mutual rotation between the plates 41 and 42.

The clutch may comprise an adjuster configured to vary the clamping force between plates 41 and 42 and thus the maximum torque. Said adjuster may vary the clamping force between plates 41 and 42 and thus the maximum torque depending on the compression of spring 44.

Optionally, the clutch may provide a manually operated configuration change. Alternatively or additionally, the configuration variation may be automatically controlled for example given by the below described means for controlling and/or function of the angular opening between the attachments 2 and 3 and thus between the prostheses 1a and 1b. Alternatively, the change in configuration may be automatically controlled and the clutch may comprise a cam 47 configured to command a move away between the plates 41 and 42 (and thus a move into a free configuration) when the angular opening between the attachments 2 and 3 defines an angle almost at least equal to a threshold value. Accordingly, when said angular opening is almost at least equal to a threshold value the cam 47 moves the plates 41 and 42 away from each other as opposed to the spring 44, whereas when said angular opening is almost less than the threshold value the cam 47 does not act on the plates 41 and 42 which are tightened between them by the spring 44.

This threshold value beyond which the cam configured to command a departure between plates 41 and 42 can be basically between 90° and 270° and in detail between 120° and 210° and for example almost 180°.

In detail, as shown in FIGS. 7a-7b, the cam 47 may be an axial cam i.e. configured to control a move away between the plates 41 and 42 along the axis of rotation 4a. The cam 47 may comprise a plug 47a at least integral with a plate (preferably the first plate 41); and a ramp 47b integral with a coupling (preferably the first coupling 2).

The ramp 47b has a partial (only partial) development along rotation axis 4a. It therefore defines a runway for plug 47a transverse to axis 4a.

In particular, the pin 47a, being integral with the first plate 41, rotates dragged by the first plate 41. When said angular opening is substantially at least equal to the threshold value, the plug 47a meets the ramp 47b and rises on it. At this point the pin 47a, sliding on the transversal track to the axis 4a defined by the ramp 47b, translates along the rotation axis 4a causing the reciprocal detachment of the plates 41 and 42 and therefore the passage from the operating configuration (FIG. 7a) to the free configuration (FIG. 7b).

The mover 5 can be bound at least partially to the first attachment 2.

It can command a relative rotation between first attachment 2 and second attachment 3.

The mover 5 (FIG. 4) may comprise a motor 51 and a transmission 52 configured to transmit the outgoing motion from the motor 51 to the hinge 4 by defining the reciprocal rotation between the connections 2 and 3.

The transmission 52 may be of a mechanical type and, for example, comprise a first wheel 521 driven by the motor 51, a second wheel 522 configured to be at the hinge 4 and at least one element 523 transmitting motion from the first wheel 521 to the second wheel 522.

The first wheel 521 can have its axis of rotation almost parallel to axis 4a.

The first wheel 521 can be hinged to the first attachment 2.

The second wheel 522 may have an axis of rotation substantially coincident with the axis of rotation 4a.

The second wheel 522 can be integral with the hinge 4 and, to be precise, with the first plate 41 and therefore with the second attachment 3 via the clutch. In detail, in an operating configuration, the second wheel 42 discharges torque on the first plate 41 which, being in contact with the second plate 42, drags said second plate 42 in rotation generating between the plates 41 and 42, and therefore between the attachments 2 and 3, a handling torque. The driving torque is preferably less than said maximum torque transmissible by the transmission.

It should be noted that if the clutch does not allow torque to pass between the plates 41 and 42 (e.g. in a free configuration or with handling torque greater than the maximum transmissible torque), the second wheel 522 pulls the first plate 41 which, however, does not transmit torque to the second plate 42. In this case, the mover 5 does not act on the prosthesis 1 and the second wheel 522 and first plate 41 assembly rotates with respect to the rest of the prosthesis 1 without causing rotation between the attachments 2 and 3.

The element 523 may be a belt and the wheels 521 and 522 may be pulleys.

Preferably, the element 523 may be a toothed belt and the wheels 521 and 522 may be sprockets.

The motor 51 can be electric and in particular a servomotor.

It can be seen that the motor 51 can define an output defining an additional axis of rotation substantially transverse and in detail nearly normal to the axis of rotation 4a. Therefore, the mover 5 may comprise an additional transmission 53 interposed between the motor 51 and the transmission 52.

The additional transmission 53 may comprise an endless screw 531 integral with the output and configured to rotate about said additional axis of rotation, and a sprocket 532 integral with the first wheel 521 so as to rotate about an axis of rotation nearly parallel to the axis of rotation 4a and configured to take motion from said screw 531.

Joint prosthesis 1 may include a compensator 6 configured to apply a locking torque to the hinge defining the minimum torque to be applied to hinge 4 to determine reciprocal rotation between attachments 2 and 3.

The joint prosthesis 1 can thus define a minimum torque (essentially equal to the locking torque) necessary to have a rotation between the attachments 2 and 3 and preferably a maximum torque beyond which there is no rotation due to transition into free configuration. Therefore, in order to have said rotation, the mover 5 (or an element external to the prosthesis such as gravity or the action of, for example, an object) will have to apply a driving torque greater than said minimum torque and appropriately less than the maximum torque.

The locking torque, as further described below, is variable as a function of the angular opening between connections 2 and 3. In particular, it is proportional and to be precise directly proportional to the angular opening and thus to the angle between connections 2 and 3.

The compensator 6 may comprise a cable 61 having a first end integral with the first attachment 2 and a second end integral with the second attachment 3 and a pulley 62 for sliding the cable 61.

The cable 61 can be made of metallic material (e.g. steel) or synthetic fibre such as Gel Spun Polyethylene (also known as Dyneema).

The cable 61 has a first end integral with the first attachment 2 and a second end integral with the second attachment 3 so as to have a movement of the cable 61 at a reciprocal rotation of the attachments 2 and 3 and advantageously a variation of the tension of the cable 61 and therefore of the locking torque.

In particular, the compensator 6 may comprise a first constraint 63 defining a first anchoring point of the first end of the cable 61 to the first attachment 2 and a second constraint 64 defining a second anchoring point of the second end of the cable 61 to the first attachment 2 to the second attachment 3 and more precisely to the first plate 41 and/or the second wheel 522. Furthermore, the compensator 6 includes an additional deflection pulley 68 interposed between the first constraint 63 and the pulley 62 and configured to ensure the correct angle of winding of the cable 61 on the pulley 62.

The additional pulley 68 can define for cable 61 an intermediate constraint to constraints 61 and 62.

The additional pulley 68 can be hinged to the second attachment 3.

The additional pulley 68 can be at a radial distance from the axis of rotation 4a allowing the cable tension 61 to generate said locking torque.

This radial distance can be substantially between 20 and 50 mm.

The first constraint 63 can be at a radial distance from the axis of rotation 4a. Said radial distance may be substantially between 5 and 30 mm.

In the document, the term ‘radial’ identifies a direction/axis perpendicular to the axis of rotation 4a.

This radial distance may be adjustable and thus the compensator 6 may include means of adjusting the first constraint 63, i.e. the anchor point of the first end of the cable 61 at the first attachment 2 by varying the cable tension 61.

The adjustment means are configured to vary the radial distance between the first anchor point and the axis of rotation 4a. They may comprise a slider slidable relative to the second attachment 2 along a direction normal to the axis 4a and means of resolvable locking of the slider to the second attachment 3.

It is shown how the means of adjustment can, for example, allow a tension of cable 61 such as to define a torque at the first constraint 63 and thus at the hinge 4 rebalancing the torque generated by the weight force of the first attachment 2 and, if present, of the first prosthesis 1a appropriately independently of the reciprocal position of attachments 2 and 3.

The pulley 62 can have a rotation axis normal to axis 4a.

The pulley 62 substantially defines, for the cable 61, a path with the ends proximal to the axis of rotation with respect to the same pulley 62. In particular, it has a radial distance from the axis of rotation greater than that of the ends of the cable 61 and therefore of at least one of the constraints and the additional pulley.

The pulley 62 is loosely constrained to the second attachment 3 and in detail to the first constraint 63 so as to slide and then translate defining a sliding axis 62a suitably substantially radial and in particular nearly parallel to said adjustment axis. Furthermore, the compensator 6 may comprise elastic means 67 working in opposition to a sliding of the pulley 62 along said sliding axis 62a. Accordingly, when the attachments 2 and 3 rotate with each other, there is a reciprocal motion between the ends of the cable 61 which, sliding on the pulley 62, commands a sliding/translation of the same pulley 62 along the sliding axis 62a by loading/unloading the elastic means 67 which, at the same time, vary the tension on the cable 61 defining said locking torque and therefore said minimum torque to be applied to the hinge 4 to determine the reciprocal rotation between the attachments 2 and 3.

Since it is therefore an adjustment (always acting on the first constraint 63), it will then be possible to preload the sub-introduced elastic means 67 (thus the gravity compensation mechanism) thus reducing the length of the cable 61 and thus the overall gravity torque perceived/lifted by the actuating system.

To this end, the compensator 6 may comprise at least one sliding body 65 along which the pulley 62 slides and defining said sliding axis 62a; a slider 66 sliding along said at least one body 65 and to which the pulley 62 is hinged, suitably idle; and the aforementioned elastic means 67 working in opposition to a sliding along the at least one sliding body 65 of the pulley 62 and in detail of the slider 66.

In detail, the compensator 6 has two sliding bodies 65 and the elastic means 67 comprise a coil spring (in compression detail) wound on each body 65.

The elastic means 67 are configured to work against a change in distance between pulley 53 and rotation axis 4a.

Preferably the elastic means 67 work in opposition to a coupling of the pulley 62 to the axis 4a. They are configured to keep the cable 61 taut by favouring a reduction of the angular opening between the attachments 2 and 3.

It is emphasised that the sliding of the pulley, together with a variation in the compression of the elastic means 67, causes a variation in the tension of the cable 61 and therefore in the torque exerted by it on the hinge 4. This variation allows the compensator to oppose and balance the torque generated by the weight force of the first coupling 2 (and possibly of the first prosthesis 1a) independently of the reciprocal position of the attachments 2 and 3.

The joint prosthesis 1 may include a control board for the functioning of the prosthesis.

The control board can be in data connection with mover 5 and in detail with motor 51.

The control board can be in data connection with prosthesis control means 1 configured to allow an operator to control the operation of joint prosthesis 1. The control means can include one or more EMG sensors.

The joint prosthesis 1 may include a power supply of the mover 5 (in detail of the motor only 51) and appropriately of the control board.

The power supply may include a battery.

Advantageously, the joint prosthesis 1 can be without an active brake.

The functioning of prosthesis 1 described above in structural terms is as follows.

When the operator wishes to mutually rotate the prostheses 1a and 1b, he commands, e.g. by means of an EMG sensor, the joint prosthesis 1 to mutually rotate the attachments 2 and 3 around the rotation axis 4a. For example, the operator commands an increase in the angular opening between attachments 2 and 3.

The control board commands the mover 5 to rotate the hinges 2 and 3. Therefore, the motor 51, through the additional transmission 53, drives the transmission 52, which takes the outgoing motion from the motor 51 and, through the second wheel 521, transmits it to the hinge 4, defining the mutual rotation between the attachments 2 and 3.

The rotation of the second wheel 521 and therefore of the plates 41 and 42 drags the first constraint 63 decreeing a displacement of the first end of the cable 61. This movement of said end results in a sliding of the pulley 62 along the sliding axis 62a and therefore in a compression of the elastic means 67.

As a result, the cable 61 is stretched more tautly, resulting in a greater locking torque that will hold the arm in the desired position as opposed to, for example, the copy determined by the weight force of the second attachment 3 and the second prosthesis 1b. In this condition, motor 51 can thus be advantageously deactivated. The joint prosthesis 1 according to the invention achieves important advantages.

In fact, the joint prosthesis 1 features a low perceived weight, comfortable to wear and use, a low price, a responsive and intuitive control strategy, low noise and finally a reliable battery life.

These advantages are at least partly determined by the adoption of a mechanical architecture that combines a free swing mechanism (defined by clutch) and, above all, by a particular mover 5 that is non retro-drivable with low noise and advantageously assisted by a gravity compensator 6. These aspects define a joint prosthesis 1 that is easy to control especially in comparison to known prostheses where the joint prosthesis 1 can flex unpredictably if the patient moves the upper arm close to the horizontal during free swing mode due to the inability to counter gravity.

The joint prosthesis 1 is also preferable because, in contrast to known joint prostheses using active brakes combined with an active drive unit, it does not have a brake (resulting in high current consumption) or a rear-mounted gearbox with an often complex and fragile passive brake at the gearbox input. In fact, in joint prosthesis 1, the only torque that mover 5 (i.e. motor 51) has to provide is that required to overcome clutch resistance, the inertia of the elbow prosthesis, plus any gravity torque created by a load held by a prosthetic hand. Consequently, the joint prosthesis 1 is, for example, capable of repeatedly lifting heavy objects with reduced power and cost compared to known prostheses.

Other advantages of the joint prosthesis 1 are the immediate response to user input, the possibility of a silent and natural-looking free swing of the prosthesis 1, an anthropomorphic appearance and functionality similar to that of a real joint.

The invention is susceptible to variations within the scope of the inventive concept as defined by the claims. Within that scope, all details are substitutable by equivalent elements and the materials, shapes and dimensions can be any.

1. Joint prosthesis configured to reciprocally rotate a first prosthesis and a second prosthesis and comprising:
a first attachment of said joint prosthesis to said first prosthesis;
a second attachment of said joint prosthesis to said second prosthesis;
a hinge defining an axis of rotation between said attachments and thus between said prostheses;
a mover configured to apply to said hinge a driving torque defining a reciprocal rotation between said attachments and thus between said prostheses about said axis of rotation;
characterised by comprising
a compensator configured to apply a locking torque to said hinge defining the minimum torque to be applied to said hinge to determine said reciprocal rotation between said connections;
by that
said locking torque is variable depending on the angular opening between said connections;
and the fact that said compensator comprises
a cable having a first end integral with said first attachment and a second end integral with said second attachment,
a pulley of said cable constrained in a compliant way to said second attachment so as to slide defining a sliding axis substantially radial to said axis of rotation;
elastic means working against a sliding of said pulley and said slider along said sliding axis.

2. Joint prosthesis according to claim 1, wherein said compensator comprises at least one sliding body along which said pulley slides; a slider sliding along said sliding body and to which said pulley is hinged; and wherein said elastic means working in opposition to a said sliding along said sliding body of said pulley and said slider.

3. Joint prosthesis according to claim 2 wherein said elastic means comprise a compression spring.

4. Joint prosthesis according to claim 1, wherein said compensator comprises adjustment means configured to vary the radial distance of the first anchor point of said cable to said first attachment from said axis of rotation.

5. Joint prosthesis according to claim 1, wherein said cable is made of synthetic fibre.

6. Joint prosthesis according to claim 1, wherein said pulley defines for said cable a path substantially with the ends of said cable proximal to said axis of rotation with respect to said pulley.

7. Joint prosthesis according to claim 1, wherein said hinge comprises a clutch defining a maximum torque transmissible from said mover to said second attachment; wherein said clutch comprises a first plate constrained to said second attachment; a second plate constrained to said mover; at least one guide defining a sliding axis between said plates parallel to said axis of rotation, and a spring configured to define along said sliding axis a reciprocal clamping force between said plates defining said maximum torque.

8. Joint prosthesis according to claim 1, wherein said clutch defines an idle rotation between said plates for an angle not exceeding an idle angle substantially between 15° and 10°; wherein said first plate is between said second plate and said second attachment; wherein said clutch comprises at least one connector of said second plate to said second attachment; wherein said connector is a pin defining said guide; and wherein said first plate comprises, for cach of said at least one connector, at least one slot having an angular width substantially equal to said idle angle so as to allow said second plate to rotate with respect to said first plate through said idle angle by dragging said connector along said slot.
